(19) United States
(12) Patent Application Publication
(10) Pub. No.: US 2006/0029556 A1
(43) Pub. Date: Feb. 9, 2006

(54) Aqueous-alcoholic depigmenting gels

(75) Inventors: Fabienne Louis, Villeneuve-Loubet (FR); Sandrine Orsoni, Mandelieu (FR); Laurent Fredon, Roquefort-Les-Pins (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA RESEARCH & DEVELOPMENT, S.N.C., VALBONNE SOPHIA ANTIPOLIS (FR)

(21) Appl. No.: 11/150,176

(22) Filed: Jun. 13, 2005

Related U.S. Application Data
(63) Continuation of application No. PCT/EP03/15021, filed on Dec. 3, 2003.
(60) Provisional application No. 60/434,433, filed on Dec. 19, 2002.

(30) Foreign Application Priority Data
Dec. 12, 2002 (FR) 02/15750

(51) Int. Cl. A61K 8/34

(52) U.S. Cl. 424/59; 424/62

(57) ABSTRACT

Topically applicable, stable and non-irritating aqueous-alcoholic cosmetic/dermatological gels suited for cutaneous depigmentation contain water, at least one alcohol, at least one gelling agent, at least one phenolic compound, at least one retinoid, and, optionally, at least one sunscreen, formulated into a topically applicable, physiologically acceptable medium therefor.

### CROSS-REFERENCE TO PRIORITY/PCT/PROVISIONAL APPLICATIONS

This application claims priority under 35 U.S.C. § 119 of FR 02/15750, filed Dec. 12, 2002, and of provisional application Ser. No. 60/434,433, filed Dec. 19, 2002, and is a continuation of PCT/EP 2003/015021, filed Dec. 3, 2003 and designating the United States (published in the English language on Jun. 24, 2004 as WO 2004/052353 A3); each hereby expressly incorporated by reference and each assigned to the assignee hereof.

### BACKGROUND OF THE INVENTION

1. Technical Field of the Invention

The present invention relates to depigmenting compositions for cosmetic or pharmaceutical applications, comprising a phenolic compound, a retinoid, especially a dispersed retinoid, and optionally a sunscreen, formulated as aqueous-alcoholic gels.

By virtue of the composition thereof, such gels provide same with both stability and harmlessness.

2. Description of Background and/or Related and/or Prior Art

Among the therapeutic agents recommended in the treatment of cutaneous hyperpigmentation, phenolic compounds such as hydroquinone and derivatives thereof have for decades been among the active agents that are the most effective. The therapeutic use of these agents results from the observation of cutaneous depigmentation in the case of operatives in the rubber industry, in which certain of these products are used as antioxidants. Subsequently, numerous studies have confirmed their efficacy, alone or combined with other depigmenting agents [Jorge L. Sanchez, M.D., and Miguel Vazquez, M.D., International Journal of Dermatology, January-February 1982, Vol. 21, pp. 55-58]. They are thus found to be active agents that are virtually indispensable in the treatment of hyperpigmentation and are consequently present in many commercial products. Hydroquinone has been the subject of various patent application filings, and in particular U.S. Pat. No. 3,856,934 in which hydroquinone is in combination with retinoic acid and a corticoid, as a depigmenting composition.

However, the incorporation of a phenolic compound such as hydroquinone presents, inter alia, two major drawbacks.

Firstly, the degradation of formulations containing phenolic compounds such as hydroquinone, alone or in combination with other active principles, is often observed. Specifically, hydroquinone is known for its sensitivity to oxidation and to heat, resulting in reduced efficacy, rapid browning of the formulations and occasionally even demixing or phase separation of the formulation.

This problem is found to be an obstacle to obtaining compositions containing several active agents, especially a phenolic compound and a retinoid.

In the prior art, sulfite salts are conventionally used to reduce this phenomenon, but they are insufficient to overcome this drawback. They can also impair the viscosity of electrolyte-sensitive formulations and thereby result in sedimentation of the active agents (for example retinoids). Specifically, the carbomers conventionally used to provide a minimum level of viscosity are affected by the electrolytes of sulfite salts and are therefore no longer sufficient alone to allow good stability of the retinoid.

Furthermore, to accelerate their dissolution, phenolic compounds such as hydroquinone are often exposed to heat during the preparation of the composition, especially in standard emulsions, this phenomenon initiating and accelerating the browning phenomenon.

The second drawback caused by the presence of phenolic compounds such as hydroquinone, alone or in combination with other active agents, in the composition is their high irritant power.

As a result of its irritant power, hydroquinone at high concentration can give rise to post-inflammatory hypermelanosis and ochronosis phenomena.

Local irritation and dermatitis may develop after a prolonged use of hydroquinone at high concentration [“N-acetyl4S cysteaminylphenol as a new type of depigmenting agent” Jimbow K., Arch. Dermatol., 1991 October; 127 (10): 1528-1534].

Treatment with hydroquinone may be accompanied by irritation that may lead to a post-inflammatory hyperpigmentation. The incidence of the irritation depends on the hydroquinone concentration. This irritation is relatively high for 10% concentrations and reduces greatly for preparations with a 5% dose, and is considered to be virtually nonexistent at a concentration of 2% [“Les agents chimiques dépigmentants (Depigmenting chemical agents)” J P. Ortonne, Ann. Dermatol. Venerol., 1986, 113: 733-736].

The selected composition may thus play a predominant role in minimizing these effects and improving the tolerance of a composition containing two potentially irritant active principles.

Thus, need continues to exist for compositions containing a phenolic compound and a retinoid that are physically stable over time, thus ensuring that the formulation remains unchanged. Such a product must also show good cosmeticity and have little irritant nature.

### SUMMARY OF THE INVENTION

It has now surprisingly been determined that aqueous-alcoholic gels containing suitable excipients provide good results in terms of physical and chemical stability. These also provide an excellent compromise between stability, especially to temperature and oxidation, efficacy, harmlessness and cosmeticity.

A process has also been developed for manufacturing the compositions according to the invention, which may be prepared under cold conditions, without heating, thus making it possible to avoid exposing the phenolic compound to heat.

The present invention thus features depigmenting compositions comprising, formulated into a physiologically acceptable medium, a phenolic compound, a retinoid, especially a dispersed retinoid, and optionally a sunscreen, characterized in that same are aqueous-alcoholic gels.

### DETAILED DESCRIPTION OF BEST MODE AND SPECIFIC/PREFERRED EMBODIMENTS OF THE INVENTION

The term “aqueous-alcoholic gel” means an aqueous gel containing alcohol and at least one gelling agent, and optionally containing a small proportion (up to 15%) of fatty phase.

All proportions are expressed as weight percentages relative to the total weight of the composition.

The compositions according to the invention preferably contain from 1% to 30% of alcohol, preferably from 2% to 20% and more particularly from 4% to 15% of alcohol.

Among the alcohols that may be mentioned, in a non-limiting manner, are ethanol, isopropanol and butanol.

The compositions according to the invention may also preferably contain one or more of the following ingredients:
a) a carbomer,
b) another gelling agent,
c) an antioxidant,
d) a chelating agent.

The compositions according to the invention of aqueous-alcoholic gel type offer good skin tolerance. They are also easier to spread than a viscous emulsion and leaves a pleasant sensation of freshness.

More particularly, this invention features aqueous-alcohol gels for depigmenting purposes, comprising one or more of the following ingredients:
from 0.01% to 10% of a phenolic compound,
from 0.0001% to 5% of a retinoid,
from 0 to 30% of sunscreens,
from 0.01% to 10% of carbomer and/or other gelling agents,
from 0.01% to 2% of antioxidants, and
from 0.01% to 1% of chelating agent.

A preferred composition according to the invention comprises:
4.00% of phenolic compound,
0.10% of retinoid,
20.00% of ethanol,
0.40% of carbomer,
0.60% of another gelling agent,
0.40% of sulfite salts,
0.10% of EDTA.

A particularly preferred composition according to the invention comprises:
4.00% of 4-hydroxyanisole,
0.10% of retinoid,
5.00% of ethanol,
0.60% of carbomer,
0.40% of another gelling agent,
0.40% of sulfite salts,
0.10% of EDTA.

Among the carbomers, non-limiting examples that may be mentioned include Carbopol 981 and Carbopol ETD 2020, sold by BF Goodrich.

Among the other possible gelling agents, non-limiting examples that may be mentioned include xanthan gum such as Keltrol T sold by Kelco, acrylate/C10-C30 alkyl acrylate crosspolymer such as the product sold under the name Pemulen TR1 or Carbopol 1382 by BF Goodrich, hydroxypropylcellulose, such as the product sold under the name Natrosol HHX 250 by Aqualon, and acrylamide/sodium acryloyldimethyltaurate copolymer and isohexadecane and polysorbate 80, sold under the name Simulgel 600 by SEPPIC.

Among the antioxidants, non-limiting examples that may be mentioned include ascorbic acid and its salts, tocopherols and sulfite salts such as sodium metabisulfite or sodium sulfite.

Examples of chelating agents that may be mentioned include ethylenediaminetetraacetic acid (EDTA), calcium disodium edetate and sodium edetate.

Phenolic compounds that may be mentioned, in a non-limiting manner, include hydroquinone, 4-hydroxyanisole and hydroquinone monobenzyl ether.

The term “retinoid” means any compound that binds to the retinoic acid receptors (RARs) and/or to retinoic X receptors (RXRs), and also precursors and derivatives thereof.

Preferably, the retinoid is a compound selected from the family of benzonaphthalene-based retinoids as described in EP-O-199,636. Adapalene (6-[3-(1-adamantyl)-4-methoxyphenyl]-2-naphthoic acid) and precursors and/or derivatives thereof will be preferred in particular. Tretinoin and isotretinoin may also be used.

The term “retinoid precursors” means the immediate biological precursors or substrates thereof, and also chemical precursors thereof.

The term “retinoid derivatives” means both the metabolic derivatives thereof and the chemical derivatives thereof.

The term “sunscreens” means a chemical sunscreen or a physical sunblock and mixtures thereof; non-limiting examples that may be mentioned include physical sunblocks such as titanium dioxide and zinc oxide, and chemical sunscreens such as octocrylene, ethylhexyl methoxycinnamate, octyl salicylate, avobenzone, oxybenzone, ecamsule and drometrizole trisiloxane.

Each sunscreen may be added at a concentration ranging from 0.001% to 20% by weight relative to the total weight of the composition.

Needless to say, the amount of the active agents in the composition according to the invention will depend on the combination selected and thus particularly on the quality of the desired treatment.

The compositions may also comprise additives usually included in cosmetics or pharmaceuticals, such as a neutralizer, a humectant and/or co-solvent, an emollient, a calmative, a preserving agent or a pH corrector, or mixtures thereof.

Too, one skilled in this art will take care to select this or these additional compound(s), and/or the amount thereof, such that the advantageous properties of the compositions according to the invention are not, or are not substantially, adversely affected.

These additives may be present in the composition in a proportion of from 0.001% to 20% by weight relative to the total weight of the composition.

Examples of neutralizers that may be mentioned include an amine base such as triethanolamine, diethanolamine or tromethamine.

An example of a pH corrector that may be mentioned is citric acid.

Examples of humectants and/or co-solvents that may be mentioned include glycerol, sorbitol, propylene glycol and macrogol 400.

The compositions according to the invention may also contain a fatty phase in a proportion ranging from 0.01% to 15%, comprising essentially an emollient. Non-limiting examples of emollients that may be mentioned include a mineral oil such as Primol 352, Marcol 82, Marcol 172 and Marcol 352 sold by Esso; a plant oil such as sweet almond oil, palm oil, soybean oil, sesame seed oil, sunflower oil, an ester such as cetearyl isononanoate, for instance the product sold under the name Cetiol SN by Cognis France, diisopropyl adipate, for instance the product sold under the name Ceraphyl 230 by ISF, isopropyl palmitate, for instance the product sold under the name Crodamol IPP by Croda, caprylic/capric triglyceride, such as Miglyol 812 sold by Huls/Lambert Riviere; a silicone oil such as a dimethicone, for instance the product sold under the name Dow Corning 200 Fluid, or a cyclomethicone, for instance the product sold under the name Dow Corning 244 Fluid by Dow Corning.

Non-limiting examples of calmatives that may be mentioned include allantoin and talc.

Examples of preserving agents that may be mentioned include benzalkonium chloride, phenoxyethanol, benzyl alcohol, diazolidinylurea and parabens, or mixtures thereof.

The present invention also features the compositions as described above, as a medicinal products.

This invention also features a process, performed at room temperature, for preparing a composition of aqueous-alcoholic gel type, comprising the following steps:
a) the preparation of the formulation phase comprising the water, the gelling agents and optionally the chelating agent, which are maintained under stirring until the mixture is totally homogeneous;
b) optionally the introduction of the neutralizer solution into the formulation phase;
c) the preparation of a first active phase comprising the phenolic compound and the alcohol, which is stirred until dissolution is complete;
d) the preparation of a second active phase comprising the retinoid and optionally the humectant, which is stirred until a smooth, homogeneous dispersion is obtained;
e) the mixing of the various active phases above into the formulation phase independently, with stirring until fully incorporated.

In a preferred embodiment, the present invention also features a process, performed at room temperature, for preparing a composition of aqueous-alcoholic gel type, successively comprising the following steps:
a) the preparation of the formulation phase comprising the water, the chelating agent and the gelling agents, which are maintained under stirring until the mixture is totally homogeneous;
b) the introduction of the neutralizer solution into the formulation phase;
c) the preparation, in a separate beaker, of a first active phase comprising the phenolic compound and the alcohol, which is stirred magnetically until dissolution is complete;
d) the preparation, in a separate beaker, of a second active phase comprising the retinoid and the humectant, which is stirred until a smooth, homogeneous dispersion is obtained;
e) the mixing of the various active phases above into the formulation phase independently, with stirring until fully incorporated.

The checking of the native pH of the mixture and the correction, where necessary, with a solution of a neutralizer, and the incorporation of the optional additives may be performed, depending on their chemical nature, during one of the steps of the preparation process described above.

Thus, in one particular embodiment of the process according to the present invention, antioxidants predissolved in water are introduced into the formulation phase after step (b).

In a last specific embodiment of the process of the invention, a fatty phase is introduced into the gel obtained after step (e).

Depending on the physicochemical characteristics of the sunscreen, one skilled in this art will take care to incorporate the sunscreen during one of the steps defined above.

The expression “formulation phase” means the mixture of a group of ingredients introduced together into a single phase.

The term “active phase” means a formulation phase containing one or more active agents.

This invention also features a regime or regimen utilizing the novel compositions as described above in cosmetics and dermatology.

The compositions of the invention are particularly suitable for treating and/or preventing dermatological complaints, conditions or afflictions associated with pigmentation disorders such as melasma, chloasma, lentigines, senile lentigo, vitiligo, freckles, post-inflammatory hyperpigmentations caused by an abrasion, a burn, a scar, a dermatosis or a contact allergy; nevi, genetically determined hyperpigmentation, hyperpigmentation of metabolic or medicational origin, melanomas or any other hyperpigmentary lesions.

The compositions according to the invention also find an application in cosmetics, in particular for preventing and/or combating the harmful effects of sunlight and/or for combating photo-induced or chronological aging of the skin and the integuments.

The compositions according to the invention are also useful in body and hair hygiene.

The present invention also features a non-therapeutic cosmetic treatment process (regime or regimen) for beautifying the skin and/or enhancing its surface appearance, wherein an aqueous-alcoholic gel comprising a phenolic compound, a retinoid, especially a dispersed retinoid, and optionally a sunscreen is topically applied onto the skin and/or its integuments.

In order to further illustrate the present invention and the advantages thereof, the following specific examples are given, it being understood that same are intended only as illustrative and in nowise limitative. Examples illustrating the stability of the compositions according to the invention are also described.

### FORMULATION EXAMPLES

In the compositions below (Examples 1 to 6), the proportions of the various constituents are expressed as weight percentages relative to the total weight of the composition.

### Example 1

| Starting Materials | % |
|---|---|
| Hydroquinone | 4.00 |
| Adapalene | 0.10 |
| Ethanol | 20.00 |
| EDTA | 0.10 |
| Carbopol 980 | 0.40 |
| Acrylate/C10-C30 alkyl acrylate | 0.60 |
| crosspolymer | |
| Sodium metabisulfite | 0.20 |
| Sodium sulfite | 0.20 |
| Propylene glycol | 5.00 |
| Glycerol | 5.00 |
| Phenoxyethanol | 1.00 |
| Aqueous 10% tromethamine solution | 4.00 |
| Citric acid | (qs pH 5-7) |
| Purified water | qs 100 |

### Example 2

| Starting Materials | % |
|---|---|
| Hydroquinone | 4.00 |
| Adapalene | 0.10 |
| Ethanol | 20.00 |
| EDTA | 0.10 |
| Carbopol 980 | 0.30 |
| Carbopol 981 | 0.30 |
| Xanthan gum | 0.40 |
| Sodium metabisulfite | 0.20 |
| Sodium sulfite | 0.20 |
| Phenoxyethanol | 1.00 |
| Propylene glycol | 5.00 |
| Glycerol | 5.00 |
| Aqueous 10% tromethamine solution | 4.00 |
| Citric acid | (qs pH 5-7) |
| Purified water | qs 100 |

### Example 3

| Starting Materials | % |
|---|---|
| Hydroquinone | 4.00 |
| Adapalene | 0.10 |
| Ethanol | 20.00 |
| EDTA | 0.10 |
| Carbopol 980 | 0.60 |
| Xanthan gum | 0.40 |
| Sodium metabisulfite | 0.20 |
| Sodium sulfite | 0.20 |
| Phenoxyethanol | 1.00 |
| Propylene glycol | 5.00 |
| Glycerol | 5.00 |
| Aqueous 10% tromethamine solution | 4.00 |
| Citric acid | (qs pH 5-7) |
| Purified water | qs 100 |

### Example 4

| Starting Materials | % |
|---|---|
| Hydroquinone | 2.00 |
| Tretinoin | 0.10 |
| Ethanol | 30.00 |
| Sodium edetate | 0.10 |
| Carbopol 981 | 0.50 |
| Carbopol 1382 | 0.50 |
| Sodium metabisulfite | 0.20 |
| Sodium sulfite | 0.20 |
| Propylene glycol | 5.00 |
| Glycerol | 5.00 |
| Triethanolamine | (qs pH 5-7) |
| Purified water | qs 100 |

### Example 5

| Starting Materials | % |
|---|---|
| 4-Hydroxyanisole | 5.00 |
| Tretinoin | 0.10 |
| Ethanol | 5.00 |
| Calcium disodium edetate | 0.10 |
| Carbopol ETD 2020 | 0.40 |
| Hydroxypropylcellulose | 1.00 |
| Sodium metabisulfite | 0.20 |
| Sodium sulfite | 0.20 |
| Propylene glycol | 5.00 |
| Macrogol E400 | 5.00 |
| Aqueous 10% tromethamine solution | 4.00 |
| Citric acid | (qs pH 5-7) |
| Purified water | qs 100 |

### Example 6

| Starting Materials | % |
|---|---|
| Hydroquinone | 4.00 |
| Adapalene | 0.10 |
| Ethanol | 20.00 |
| EDTA | 0.10 |
| Carbopol 981 | 0.60 |
| Xanthan gum | 0.40 |
| Sodium metabisulfite | 0.20 |
| Sodium sulfite | 0.20 |
| Liquid paraffin | 10.00 |
| Phenoxyethanol | 1.00 |
| Propylene glycol | 5.00 |
| Glycerol | 5.00 |
| Aqueous 10% tromethamine solution | 4.00 |
| Citric acid | (qs pH 5-7) |
| Purified water | qs 100 |

### Example 7

| Starting Materials | % |
|---|---|
| Hydroquinone | 4.00 |
| Adapalene | 0.10 |
| Ethanol | 20.00 |
| EDTA | 0.10 |
| Carbopol 1382 | 0.60 |
| Xanthan gum | 0.40 |
| Sodium metabisulfite | 0.20 |
| Sodium sulfite | 0.20 |
| Liquid paraffin | 10.00 |
| Avobenzene | 2.00 |
| Titanium dioxide | 2.00 |
| Phenoxyethanol | 1.00 |
| Propylene glycol | 5.00 |
| Glycerol | 5.00 |
| Aqueous 10% tromethamine solution | 4.00 |
| Citric acid | (qs pH 5-7) |
| Purified water | qs 100 |

### Example 8

| Starting Materials | % |
|---|---|
| Mequinol | 5.00 |
| Tretinoin | 0.10 |
| Ethanol | 15.00 |
| Calcium disodium edetate | 0.10 |
| Carbopol ETD 2020 | 0.40 |
| Hydroxypropylcellulose | 1.00 |
| Ecamsule | 1.00 |
| Sodium metabisulfite | 0.20 |
| Sodium sulfite | 0.20 |
| Propylene glycol | 5.00 |
| Macrogol E400 | 5.00 |
| Citric acid | (qs pH 5-7) |
| Purified water | qs 100 |

Formulation Examples 1 to 8 may be applied once or twice a day until total depigmentation is achieved, for the treatment of lentigines, chloasma or melasma.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable, stable and non-irritating aqueous-alcoholic cosmetic/dermatological gel suited for cutaneous depigmentation, comprising water, at least one alcohol, at least one gelling agent, at least one phenolic compound, at least one retinoid, and, optionally, at least one sunscreen, formulated into a topically applicable, physiologically acceptable medium therefor.

2. The topically applicable aqueous-alcoholic gel as defined by claim 1, comprising from 1% to 30% of alcohol.

3. The topically applicable aqueous-alcoholic gel as defined by claim 2, comprising ethanol.

4. The topically applicable aqueous-alcoholic gel as defined by claim 1, comprising at least two gelling agents and further comprising at least one carbomer, at least one antioxidant and at least one chelating agent.

5. The topically applicable aqueous-alcoholic gel as defined by claim 1, comprising about:
4.00% of phenolic compound,
0.10% of retinoid,
20.00% of ethanol,
0.40% of carbomer,
0.60% of another gelling agent,
0.40% of sulfite salts,
0.10% of EDTA.

6. The topically applicable aqueous-alcoholic gel as defined by claim 1, said at least one phenolic compound comprising hydroquinone.

7. The topically applicable aqueous-alcoholic gel as defined by claim 1, said at least one phenolic compound comprising 4-hydroxyanisole.

8. The topically applicable aqueous-alcoholic gel as defined by claim 7, comprising about:
4.00% of 4-hydroxyanisole,
0.10% of retinoid,
5.00% of ethanol,
0.60% of carbomer,
0.40% of another gelling agent,
0.40% of sulfite salts,
0.10% of EDTA.

9. The topically applicable aqueous-alcoholic gel as defined by claim 1, said at least one retinoid comprising adapalene.

10. The topically applicable aqueous-alcoholic gel as defined by claim 1, said at least one retinoid comprising tretinoin and/or isotretinoin.

11. The topically applicable aqueous-alcoholic gel as defined by claim 1, comprising about:
0.01% to 10% of a phenolic compound,
0.0001% to 5% of a retinoid,
0 to 30% of sunscreens,
0.01% to 10% of carbomer and/or other gelling agents,
0.01% to 2% of antioxidants, and
0.01% to 1% of chelating agent.

12. The topically applicable aqueous-alcoholic gel as defined by claim 1, comprising at least one chemical sunscreen and/or physical sunblock.

13. The topically applicable aqueous-alcoholic gel as defined by claim 12, comprising at least one sunscreen/sunblock selected from the group consisting of titanium dioxide, zinc oxide, octocrylene, ethylhexyl methoxycinnamate, octyl salicylate, avobenzone, oxybenzone, ecamsule and drometrizole trisiloxane.

14. A regime or regimen for treating/preventing a dermatological complaint, condition or affliction associated with a pigmentation disorder, comprising topically applying onto the affected skin area of an individual in need of such treatment, a thus effective amount of a topically applicable, stable and non-irritating aqueous-alcoholic cosmetic/dermatological gel comprising water, at least one alcohol, at least one gelling agent, at least one phenolic compound, at least one retinoid, and, optionally, at least one sunscreen, formulated into a topically applicable, physiologically acceptable medium therefor.

15. A regime or regimen for preventing and/or combating the harmful effects of sunlight and/or for combating photo-induced or chronological aging, comprising topically applying onto the affected skin area of an individual in need of such treatment, a thus effective amount of a topically applicable, stable and non-irritating aqueous-alcoholic cosmetic/dermatological gel comprising water, at least one alcohol, at least one gelling agent, at least one phenolic compound, at least one retinoid, and, optionally, at least one sunscreen, formulated into a topically applicable, physiologically acceptable medium therefor.

16. A regime or regimen for beautifying the skin and/or integuments thereof and/or enhancing its/their surface appearance, comprising topically applying onto the affected skin/integument area of an individual in need of such treatment, a thus effective amount of a topically applicable, stable and non-irritating aqueous-alcoholic cosmetic/dermatological gel comprising water, at least one alcohol, at least one gelling agent, at least one phenolic compound, at least one retinoid, and, optionally, at least one sunscreen, formulated into a topically applicable, physiologically acceptable medium therefor.

17. A process for the preparation of the topically applicable aqueous-alcoholic gel as defined by claim 1, comprising conducting the following steps essentially at room temperature:
a) preparing a formulation phase which comprises water, the gelling agent(s) and optionally a chelating agent, which are maintained under stirring until the mixture is totally homogeneous;
b) optionally, introducing a neutralizer solution into the formulation phase;
c) preparing a first active phase which comprises the phenolic compound and the alcohol, which is stirred until dissolution is complete;
d) preparing a second active phase which comprises the retinoid and optionally a humectant, which is stirred until a smooth, homogeneous dispersion is obtained;
e) mixing the various active phases into the formulation phase independently, with stirring until fully incorporated.

18. The process as defined by claim 17, comprising introducing antioxidants predissolved in water into the formulation phase after step (b).

19. The process as defined by claim 17, comprising introducing a fatty phase into the gel obtained after step (e).
